(19) United States
(12) Patent Application Publication
(10) Pub. No.: US 2025/0318746 A1
(43) Pub. Date: Oct. 16, 2025

(54) SYSTEM AND METHOD FOR ASSESSING RESPIRATION

(71) Applicant: Reflexion Interactive Technologies, Inc., Lancaster, PA (US)

(72) Inventors: Joseph Terranova, New Berlin, WI (US); George Kong, Ephrata, PA (US); Matthew Campagna, Lancaster, PA (US); Tyler Bennett, Arlington, VA (US)

(21) Appl. No.: 19/177,242

(22) Filed: Apr. 11, 2025

Related U.S. Application Data
(60) Provisional application No. 63/632,777, filed on Apr. 11, 2024.

(51) Int. Cl. A61B 5/08; A61B 5/00; A61B 5/11; A61B 5/113

(52) U.S. Cl. CPC A61B 5/0803; A61B 5/1128; A61B 5/1135; A61B 5/7405; A61B 5/7445; A61B 5/7455; A61B 2562/0204

(57) ABSTRACT

A novel and advantageous system and method for assessing respiration is provided. Particularly, a novel and advantageous system and method for tracking and assessing respiration is provided. More particularly, a novel and advantageous system and method for guiding, tracking, and assessing respiration and providing real-time feedback on a user's breathing is provided.

### CROSS-REFERENCE TO RELATED APPLICATIONS

The present disclosure claims priority to Provisional Application No. 63/632,777, entitled SYSTEM AND METHOD FOR TRACKING AND ASSESSING RESPIRATION, and filed 11 Apr. 2025, the content of which is hereby incorporated by reference herein in its entirety.

### FIELD OF THE INVENTION

The present disclosure relates to a novel and advantageous system and method for assessing respiration. Particularly, the present disclosure relates to a novel and advantageous system and method for tracking and assessing respiration. More particularly, the present disclosure relates to a novel and advantageous system and method for guiding, tracking, and assessing respiration and providing real-time feedback on a user's breathing.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

“Breathwork” is the regulated control of one's breathing to achieve one or more of therapeutic, human performance, psychological, aesthetic, entertainment, or physiological benefits. During the practice of breathwork, users conform to one of a multiplicity of respiration patterns expressed in terms of one or more of: frequency, intensity, rhythm, loudness, and the movements of specific body parts, such as the nose, mouth, chest, or diaphragm. Breathwork is generally presented by a live coach or through audio or video recordings, which have the disadvantages of high cost or lack of detailed, real-time, individualized feedback.

There is a need in the art for a system and method for breathwork that can provide detailed, real-time feedback and guidance. There is further a need in the art for tracking and assessment of breathwork that can be done without the need to conform to patterns communicated by a coach or recording.

### BRIEF SUMMARY OF THE INVENTION

The following presents a simplified summary of one or more embodiments of the present disclosure in order to provide a basic understanding of such embodiments. This summary is not an extensive overview of all contemplated embodiments, and is intended to neither identify key or critical elements of all embodiments, nor delineate the scope of any or all embodiments.

The present disclosure, in one or more embodiments, relates to a method for assessing breathing including sensing input about a user's breathing over a time period, gathering data about the user's breathing, assessing the user's breathing, and providing feedback to the user regarding the user's breathing. Sensing input may comprise gathering data about the user's breathing over the time period. The data gathered may include movement of the user's head and/or thoracic region during the time period. The data may include pose, such as position and orientation of the user's head. The data may include sounds produced by the user's body, such as sounds produced by the user's mouth and/or sounds produced by the user's nose. The feedback may include a score and/or may be one of “breathe more quickly,” “breathe more slowly,” “breathe more deeply,” “extend your inhale,” or “extend your exhale.”

The method may further include providing a cue to the user prior to gathering data about the user's breathing. The cue may be a cue type such as inhale, exhale, or hold and assessing the user's breathing may include assessing compliance with the cue type. Assessing compliance with cue type may include determining a breath state of the user and comparing the breath state to the cue type. Feedback may include a score and wherein the score is calculated by dividing a percentage of breath operations performed by a user matching corresponding cues by a total number of cues.

The method may further include establishing a baseline for elements of a user's pose. In some embodiments, the method may comprise iteratively adjusting the baseline during the user's breathing.

The present disclosure, in one or more embodiments, additionally relates to a system for assessing a user's breathing. The system may include one or more pose tracking systems, a tracking module, an assessment module, and a feedback module. In some embodiments, the system may further include a microphone.

The one or more pose tracking systems may be configured for tracking breathing of a user. The one or more pose tracking systems may detect at least one of position, positional acceleration, rotation, and rotational acceleration of user's head and/or thoracic region. The one or more the pose tracking systems may be disposed in a wearable or in a virtual or augmented reality headset. The tracking module may be configured for collecting data about the user's breathing from the one or more pose tracking systems. The assessment module may be configured for assessing the user's breathing. The feedback module may be configured for providing feedback to the user regarding the user's breathing. The microphone may track audio associated with breathing of the user.

While multiple embodiments are disclosed, still other embodiments of the present disclosure will become apparent to those skilled in the art from the following detailed description, which shows and describes illustrative embodiments of the invention. As will be realized, the various embodiments of the present disclosure are capable of modifications in various obvious aspects, all without departing from the spirit and scope of the present disclosure. Accordingly, the drawings and detailed description are to be regarded as illustrative in nature and not restrictive.

### BRIEF DESCRIPTION OF THE DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying Figures, in which:

FIG. 1a illustrates a block diagram of components of a system for guided breathing, in accordance with one embodiment.

FIG. 1b illustrates a block diagram of components of a system for guided breathing, in accordance with another embodiment.

FIG. 2 illustrates three steps of a method of guided breathing, in accordance with one embodiment.

FIG. 3a illustrates a block diagram of major system components, in accordance with one embodiment.

FIG. 3b illustrates a block diagram of major system components, in accordance with another embodiment.

FIG. 3c illustrates a block diagram of major system components, in accordance with a further embodiment.

FIG. 3d illustrates a block diagram of major system components, in accordance with yet another embodiment.

FIG. 4 illustrates a screenshot showing a first object related to the desired breathing cadence and a second object related to the user's detected breathing pattern, in accordance with one embodiment.

FIG. 5a is a 3-dimensional depiction if a user wearing a virtual or augmented reality headset superimposed over x-, y-, and z-axes, in accordance with one embodiment.

FIG. 5b is a simplified 2-dimensional side view depiction of a user showing displacement of the user's head between samples, in accordance with one embodiment.

FIG. 5c is a 3-dimensional block depiction of a user showing the difference in rotation from τ−1 to τ, in accordance with one embodiment.

FIG. 6 illustrates a sinusoidal movement with noise and the same data after exponential smoothing.

FIG. 7 illustrates acoustic input from respiration in relation to head movement.

FIG. 8 illustrates a baseline tracking the neutral midpoint of periodic motion even as a large offset is introduced.

FIG. 9a illustrates four phases of motion tracked during respiration, in accordance with one embodiment.

FIG. 9b illustrates two phases of motion tracked during respiration, in accordance with one embodiment.

FIG. 10 illustrates measured head movement compared to expected change in direction between Settle-in and Settle-out phases, in accordance with one embodiment.

### DETAILED DESCRIPTION

The present disclosure relates to a novel and advantageous system and method for assessing respiration. Particularly, the present disclosure relates to a novel and advantageous system and method for tracking and assessing respiration. More particularly, the present disclosure relates to a novel and advantageous system and method for guiding, tracking, and assessing respiration and providing real-time feedback on a user's breathing. The system and method may be used, for example, to assess and track breathing regularity, apnea, and other health conditions, as well as for entertainment purposes.

The system and method described herein may be used for assessment and tracking with or without cueing. In some embodiments, the tracking and assessment of breathing is done in correlation with cues given to the user. In other embodiments, aspects of the tracking and assessment of breathing described herein may also be used without the need to conform to any patterns communicated by a coach or audio or video recording. It is to be appreciated that the terms breathing and respiration may be used interchangeably herein.

The system and method can be used to guide a user through breathwork and then provide detailed, real-time, individualized feedback on a user's responses to detailed, real-time breathwork cues without needing a human coach or instructor to be present.

A correlation exists between human respiration and movement of the thoracic region and head. Many embodiments of the present invention exploit this correlation by using measurements of head position, optionally in combination with audio or other measurements, to characterize component parts of each breath in a form useful for providing detailed individualized feedback to the user. Such feedback may be given in real-time. In some embodiments, the measurements may be made continuously during use of the system.

### System and Method Overview

The system and method track a user's breathing based on detected data. The detected data is assessed and feedback may be given. The detected data may comprise data from a pose tracking system, such as position, positional acceleration, rotation, and rotational acceleration and data from a microphone, such as audio. Pose tracking systems may comprise, in some embodiments, inertial measurement unit (IMU) sensors. In some embodiments, the pose tracking system of the invention may improve or substitute IMU derived positional and/or rotational accuracy using cameras or other sensors. The system and method provide flexible breath detection-tracking and assessing breathing across any speed or intensity without requiring a fixed rate or pattern. The system and method assess the detected data and provide feedback. The assessment looks at basic respiratory rate, but also may analyze inhale/exhale depth, pacing, and holds to provide low-latency, personalized feedback. The system may be integrated into a head-mounted wearable without additional sensors or chest straps. It is to be appreciated, however, that additional sensors or chest straps may be used if desired.

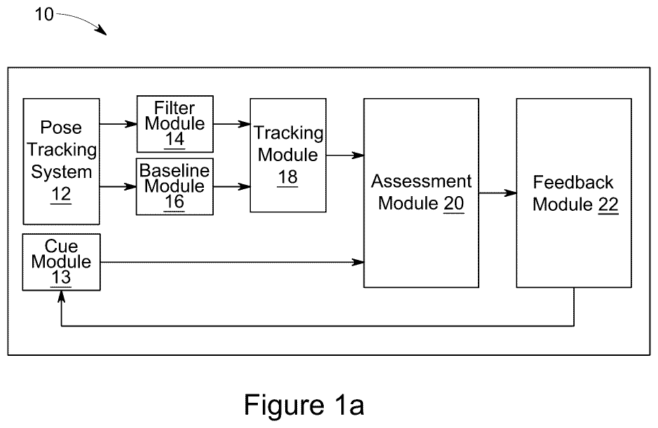

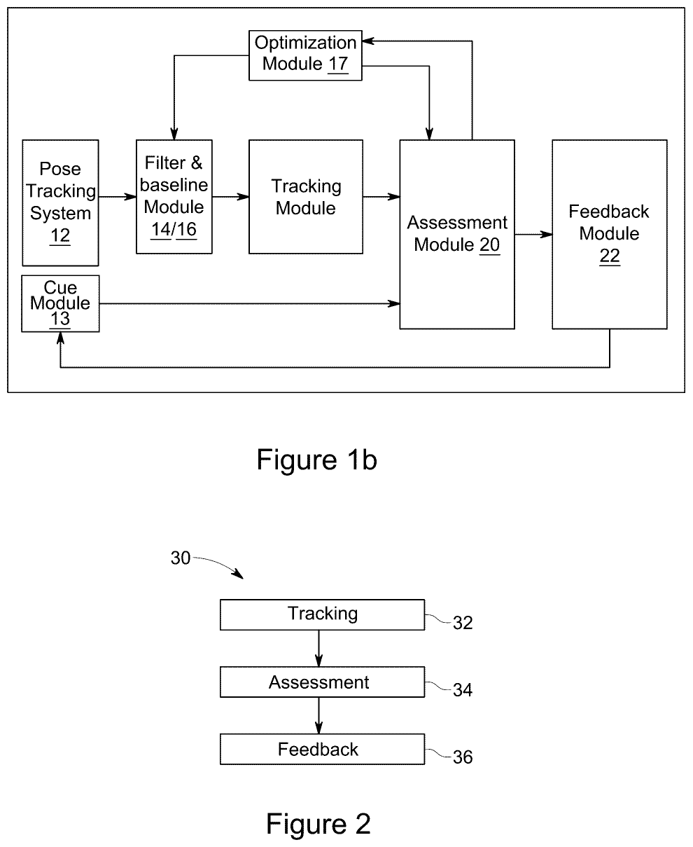

FIGS. 1a and 1b illustrate block diagrams of components of a system for assessing breathing, or guided or unguided breathing, as well as data flow through the system, in accordance with various embodiments embodiment. The system 10 includes a pose tracking system 12, a cue module 13, a filter module 14, a baseline module 16, an optimization module 17, a tracking module 18, an assessment module 20, and a feedback module 22. In some embodiments, the system further includes one or more output devices. In various embodiments, more or fewer modules of the system may be provided, one or more steps in the method may not be done, and other steps may be done. Various of the modules may be combined or omitted. In some embodiments, feedback may not loop back to cueing, no cue module may be provided, a baseline module may not be provided, and/or optimization may not be done between assessing and filtering. As shown in FIG. 1b, the system may include an optimization module looping between assessment and filter/baseline. In some embodiments, a scoring module may be included.

FIG. 2 illustrates three steps of a method 30 for assessing breathing, or guided or unguided breathing, in accordance with one embodiment. These include tracking 32, assessment 34, and feedback 36. Based on tracking 32 and assessment 34, feedback 36 may be given about a user's breathing, which may be used in a variety of ways including breathwork. Tracking 32 may include capturing sensor data and characterizing the component parts of each breath to determine breath states. Assessment 34 may compare the component parts of each breath with cues or other rule sets that define correct or desired breathing. Feedback 36 may present information to the user based on results from assessment and/or tracking. The feedback may comprise, for example, computer-graphic animation of the user's breathing. In some embodiments, the feedback may further include guidance such as detailed, real-time, individualized instructions, such as “breathe more quickly” or “breathe more deeply” based on detected characteristics of the user's breathing. In some embodiments, feedback may include summary feedback or a score. Each of tracking 32, assessment 34, and feedback 36 are explained more fully below.

In embodiments where cueing is not done, assessment 34 and feedback 36 may be done on criteria such as optimizing for lower respiratory rate, regulatory, consistency, etc. In some embodiments, cues may be given and assessment may be done based on compliance with cues. Cueing may present real-time instructions to the user about how and when to breathe. The instructions may include recorded audio, video, computer-graphic animations, or other suitable media.

In various embodiments, the method may be implemented using a variety of hardware. In general, suitable hardware includes hardware for a pose tracking system. The pose tracking system may include pose tracking sensors such as inertial measurement unit (IMU) sensors. The hardware may further include a sensor, such as a microphone, for picking up audio data. In some embodiments, the hardware may be a head-mounted wearable. For example, the method may be implemented using a virtual- or augmented-reality headset or smart glasses, an app running on a smartphone or tablet, a sleep mask, wireless earbuds, a fixed installation at a breathwork practitioner's office, or other.

IMU sensors, such as accelerometers and gyroscopes, track micro-movements of a user's head and body. The tracked micro-movements may be integrated with microphone input, or audio. The sensed micro-movements and audio (collectively, “detected data”) may be correlated with respiration.

Data processing and baseline calibration may be done on detected data. More specifically, filters may be used to process raw sensor data and establish dynamic baselines using alpha blending to track breath states (inhale, exhale, hold).

An assessment and feedback loop delivers corrective feedback to a user. Such corrective feedback may be provided through visual indicators, haptic feedback, audio prompts, and/or VR/AR overlays. In some embodiments, assessment may comprise comparing detected breathing patterns with cue instructions.

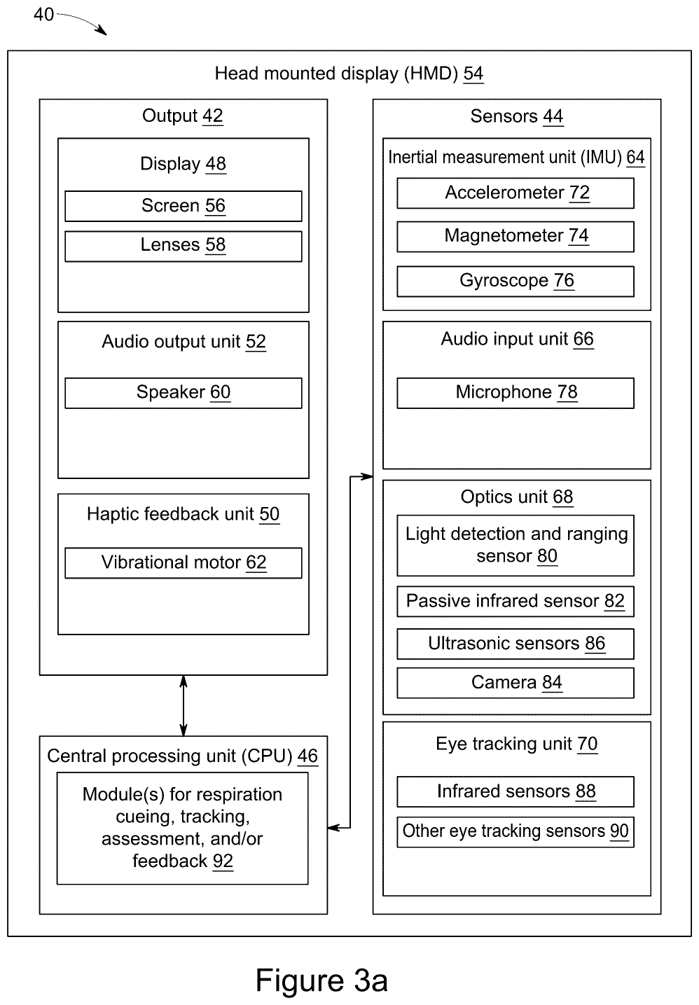

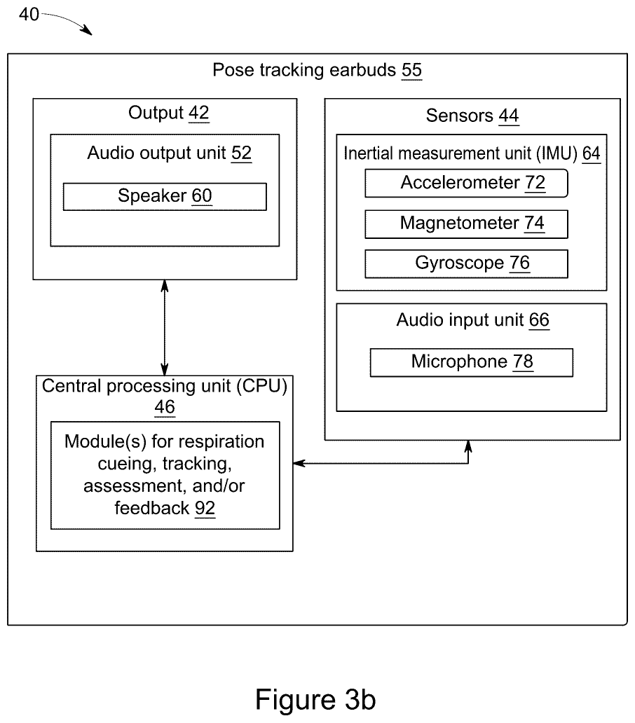

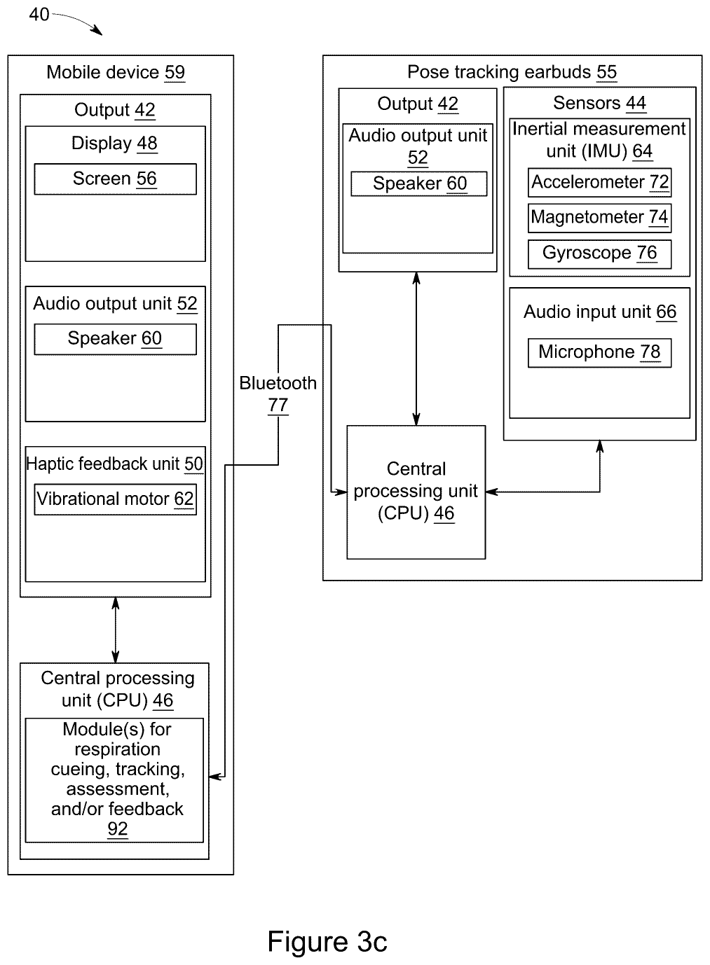

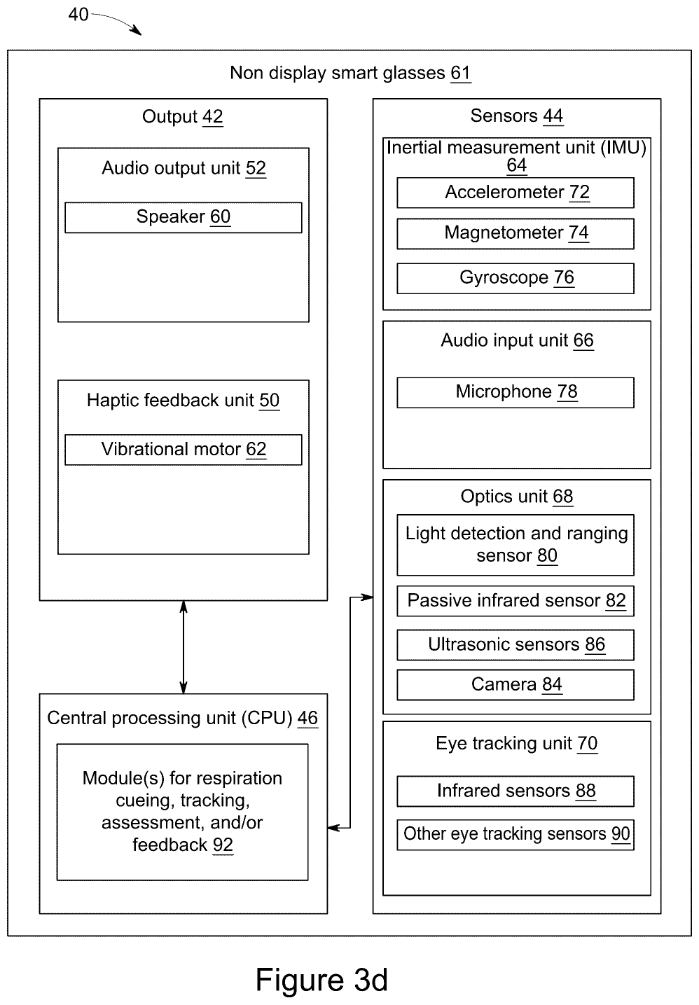

FIGS. 3a, 3b, and 3c, and 3d illustrates a block diagram of major system components of various embodiments. It is to be appreciated that any reference to a component part in the singular or in the plural is not intended to be limiting; more than one of an item referenced in the singular and only one of an item referenced in the plural may be provided. In general, the system 40 may have one or more outputs 42, one or more sensors 44, and a central processing unit (CPU) or similar device 46. The one or more outputs 42 may be, for example, a visual output 48 (such as a display), haptic feedback unit 50, and/or an audio output 52. The sensors 44 may be part of a pose tracking system.

FIG. 3a illustrates an embodiment including a head mounted display (HMD) 54. As shown, the system 40 may have one or more outputs 42, one or more sensors 44, and a central processing unit (CPU) 46 or similar device. The outputs 42 may include, for example, a display 48, an audio output unit 52, and a haptic feedback unit 50. The display 48 may be a screen and/or lenses. The audio output unit 52 may be a speaker. The haptic feedback unit 50 may be a vibrational motor. The sensors 44 may comprise one or more of an inertial measurement unit (IMU) 64, an audio input unit 66, an optics unit 68, and an eye tracking unit 70. The IMU 64 may include an accelerometer 72, a magnetometer 74, and/or a gyroscope 76. The audio input unit 66 may comprise a microphone 78 or similar device. The optics unit 68 may comprise one or more of a light detection and ranging sensor 80, a passive infrared sensor 82, a camera 84, and/or ultrasonic sensors 86. The eye tracking unit 70 may comprise infrared sensors 88 and/or other eye tracking sensors 90. The CPU 46 may include modules 92 such, as modules for one or more of respiration cueing, tracking, assessment, optimization, and feedback.

FIG. 3b illustrates a block diagram of major system components of an embodiment using pose tracking earbuds 55. As shown, the system 40 may have one or more outputs 42, one or more sensors 44, and a central processing unit (CPU) or similar device 46. The outputs 42 may be, for example, an audio output 52 such as a speaker or other suitable output device. The sensors 44 may comprise an inertial measurement unit (IMU) 64 and/or an audio input unit 66. The IMU 64 may include an accelerometer 72, a magnetometer 74, and/or a gyroscope 76. The audio input unit 66 may comprise a microphone 78 or similar device. The CPU 46 may include modules 92 such, as modules for one or more of respiration cueing, tracking, assessment, optimization, and feedback.

FIG. 3c illustrates a block diagram of major system components of an embodiment using pose tracking earbuds 57 and a mobile device 59. As shown, the mobile device 57 may have one or more outputs 42 and a central processing unit (CPU) or similar device 46. The outputs 42 may include, for example, a display 48, an audio output unit 52, and a haptic feedback unit 50. The CPU 46 may include modules 92 such, as modules for one or more of respiration cueing, tracking, assessment, optimization, and feedback. The pose tracking earbuds 57 may be substantially as described with respect to FIG. 2d. The mobile device and pose tracking earbuds may communicate therebetween, such as via Bluetooth™ connection.

FIG. 3d illustrates a block diagram of major system components of an embodiment using a non-display smart glasses 61. As shown, the system 40 may have one or more outputs 42, one or more sensors 44, and a central processing unit (CPU) or similar device 46. The outputs 42 may include, for example, an audio output unit 52, and a haptic feedback unit 50. The audio output unit 42 may be a speaker 60. The haptic feedback unit 50 may be a vibrational motor 62. The sensors 44 may comprise one or more of an inertial measurement unit (IMU) 64, an audio input unit 66, an optics unit 68, and an eye tracking unit 70. The IMU 64 may include an accelerometer 72, a magnetometer 74, and/or a gyroscope 76. The audio input unit 66 may comprise a microphone 78. The optics unit 68 may comprise one or more of a light detection and ranging sensor 80, a passive infrared sensor 82, a camera 84, and/or ultrasonic sensors 86. The eye tracking unit 70 may comprise infrared sensors 88 and/or other eye tracking sensors 90. The CPU 46 may include modules 92 such, as modules for one or more of respiration cueing, tracking, assessment, optimization, and feedback.

### Cueing

Cueing presents real-time instructions to the user about how and when to breathe. The instructions may include recorded audio, video, computer-graphic animations, and/or other suitable media. It is to be appreciated that, in some embodiments, a method for breathing assessment and feedback may not include cueing.

The types of cues given to a user may vary based on the instruction being given and based on the hardware on which the method is implemented. The cues generally can be visual, audible, tactile, haptic elements, and the like, or combinations thereof. Each cue communicates a cue operation, or type of instruction or operation for the user. A cue operation may comprise, for example, an instruction to breathe as normal, to inhale, to exhale, to hold (i.e. breath hold), to make certain sounds while breathing, or to inhale in two phases, starting with the diaphragm and ending with chest and may be categorized as different cue operation types. A cue operation may also instruct a user to employ different styles of breathing such as humming while inhaling or exhaling, as well as any other related or unrelated task.

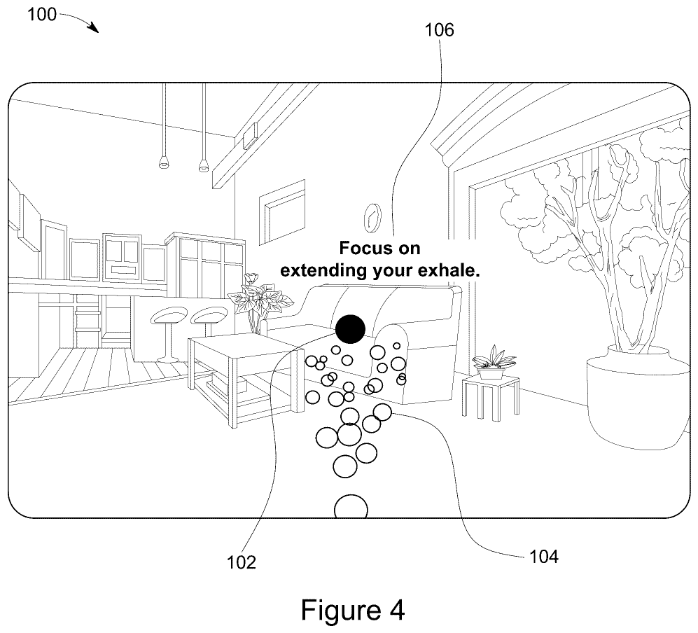

In one embodiment using visual cues, a depiction of an object is shown going away from and back towards the user. The cadence of the movement away and movement back, and optionally held at either end, correlates to the desired breathing cadence for the user. A depiction of a second object may show the user's detected breath pattern. The second object may be superimposed on the first object such that there is a visual indicator of the correlation of the user's breath pattern to the cued breath cadence. FIG. 4 illustrates a screenshot 100 showing a first object 102, such as a cue, related to the desired breathing cadence and a second object 104, such as a visual representation of breath, related to the user's detected breathing pattern, in accordance with one embodiment. In the embodiment shown, a dark cue ball 102 animates toward and away from the user cueing a breath pattern cadence. Outlined spheres 104 are shown representing the user's breath flow in and out of the mouth area. In other embodiments, different shapes, colors, or other ways of differentiating the first object from the second object may be used. While the specific example of a depiction of an object is provided, it is to be appreciated that the cue may comprise a physical object, a sound, etc. As shown in FIG. 4, text feedback 106, or guidance based on assessment, may also be provided to the user, such as to focus on extending an exhale. Some embodiments may include only a depiction of a user's detected breath pattern without any depiction of a cue. This may be useful, for example, in a firearm training application.

In some embodiments, a user can create a customized set of cue operations. Customizations include, for example, length of breath, number of breaths, length of breath hold, and the order in which the cue operations are presented to the user.

The cueing system may be used to provide input to the assessment system, which determines the degree to which the user's respiration is synchronized and compliant in other forms with the given cue operation. As previously noted, in some embodiments cueing may not be used and other compliance parameters may be used in assessment. Other forms of compliance include, but are not limited to, depth of breath, audio threshold achieved, etc.

A “breathwork practice” defines a specific ordered list of cue operations. One example of a breathwork practice is Cyclic Hyperventilation and includes cue operations to inhale for three seconds, to exhale for three seconds, and to repeat until a breath hold cue operation occurs. Another example of a breathwork practice is Stress Reduction Breathwork and includes cue operations to inhale using a two-part inhale, to exhale slowly while producing a hissing sound through the mouth, and to repeat until a breath hold cue operation occurs. A further example of a breathwork practice is Box Breathing and includes cue operations to inhale for three seconds, to hold for three seconds, to exhale for three seconds, to hold for three seconds, and to repeat. Yet another example of a breathwork practice is Diaphragmatic Breathing and includes cue operations to inhale deeply using the diaphragm and to exhale slowly. The present invention is compatible with any suitable breathwork practice. Those explicitly described are for illustrative purposes and are not exhaustive.

### Isolating Movement

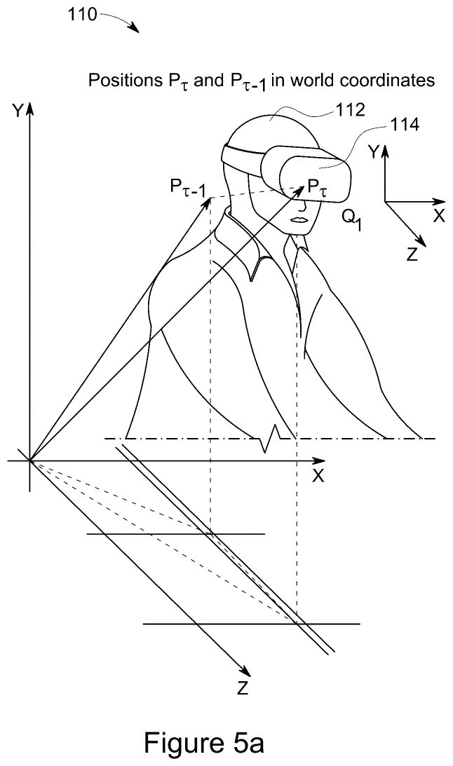

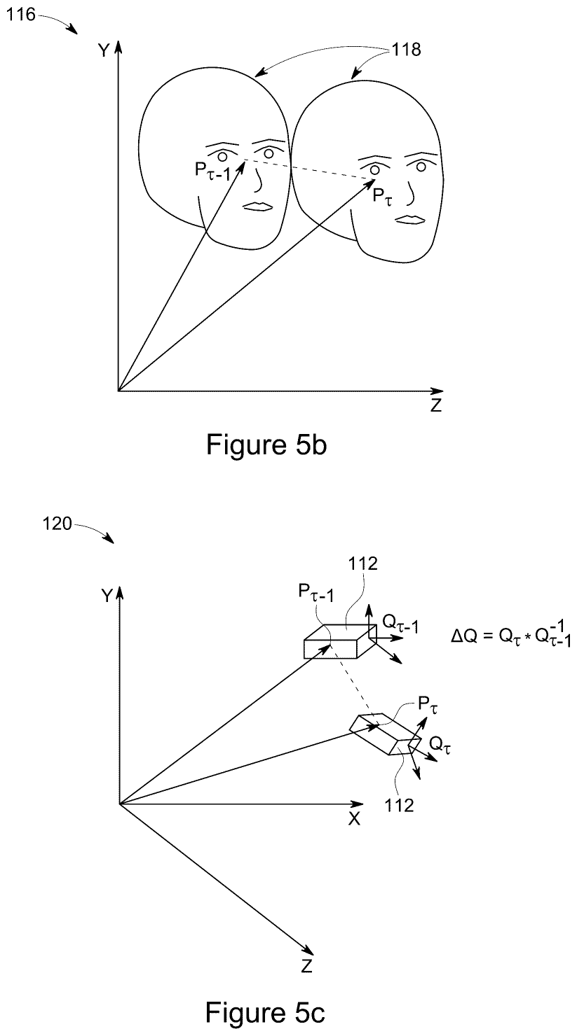

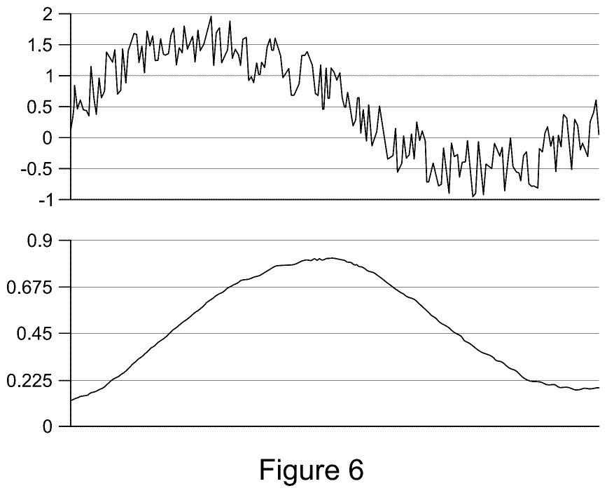

The system detects and analyzes a user's breathing patterns. In some embodiments, the system uses inertial measurement unit (IMU) sensors and audio data (sensor input) to detect a user's breathing patterns. The data may be gathered from a head-mounted wearable. FIGS. 5a, 5b, and 5c illustrate aspects of user movement during breathing and isolating such movement. This may be done after sensor input. Further detail of FIGS. 5a, 5b, and 5c is provided with discussion of systems implementing the method described herein.

FIG. 5a is a 3-dimensional depiction 110 of a user 112 wearing a virtual or augmented reality headset 114, the user 112 being superimposed over x-, y-, and z-axes. Orientation of Q τ of a local coordinate system aligned with the head such that the z-axis represents forward/backward movement, the x-axis illustrates left/right movement, and the y-axis represents up/down movement, all from the perspective of the user. The origin of the coordinate system at time τ is Pτ.

FIG. 5b is a simplified 2-dimensional side view depiction 116 of a user showing displacement of the user's head 118 between samples. FIG. 5b illustrates the change in position of the user's head 118 from time τ−1 to time τ (ΔP(Pτ−Pτ−1)).

FIG. 5c is a 3-dimensional block depiction 120 of a user 112 showing the difference in rotation from τ−1 to τ. It is to be appreciated that pitch, yaw, and roll may be accounted for in addition to movement along the x-, y-, or z-axes.

### Tracking

Tracking analyzes sensor data (sensor input). The detected data may be filtered prior to tracking. In some embodiments, a baseline may be established. While the user is breathing, the system monitors and collects data relating to measurable characteristics that correlate with breathing. These may include, for example, the position and orientation (referred to as pose) of the user's head and sounds produced by the body such as from the nose or mouth. The measured data is used in determining a breath state, as described in more detail below. In one embodiment, including cueing, the breath state may be assessed by comparing the detected data to the given cue to determine the degree to which the user's respiration is synchronized with the given cue operation, as applicable.

### Assessment

Generally, inhalation and exhalation can be divided into a plurality of windows. In one embodiment, the Assessment module of the system and method tracks a “Settle-in” phase or window, a “Settled” phase or window, and a “Settle-out” phase or window. The Settle-in phase is when a user is to begin an inhalation or exhalation. The Settled phase is when the user is following through with inhalation or exhalation. The Settle-out phase is when the user is approaching an inspiratory or expiratory pause or change in direction. It is to be appreciated that inhalation and exhalation may be otherwise divided into more or fewer windows and dynamic feedback provided based on any such windows.

### Feedback

Feedback presents information to the user based on results from the assessment. The feedback may comprise, for example, computer-graphic animation of the user's breathing. In some embodiments, the feedback may include guidance based on detected characteristics of the user's breathing. Guidance may comprise, for example, detailed, real-time, individualized instructions such as, for example, “breathe more quickly,” “breathe more slowly,” “breathe more deeply,” “extend your inhale, “extend your exhale,” and the like.

Based on the user's performance during use of the system, and, for example correlation of the user's breathing pattern to the cued breathing cadence, as applicable, an appropriate feedback response is delivered to encourage the user to continue breathing as they are or guide them into a desired rhythm. This feedback may comprise, for example, one or more of audio, iconography, text, haptics, and the like. Feedback and guidance may be given to a user in various formats. For instance, digital effects may be shown on display, including a head-mounted display (“HMD”). HMDs allow the guidance and feedback to be shown in a virtual reality or mixed reality setting.

The feedback is low-latency such that any delay between respiration by the user and the presentation of feedback, including the time it takes to perform the assessment, is negligibly perceptible to the user. Thus, the feedback can include a visual representation of the user's breath (“breath visualization”) as they are breathing that can be perceived by the user as if it were their actual breath. In addition to this low-latency feedback, summary feedback may be provided to a user.

Summary feedback may be provided at the end of a session or at any time during a session and may include measures of how closely the user's breath matched an applicable cue, breathing rate, ideal breathing rate for the individual based upon other observed physiological data (such as heart rate variability or other stress indicators), the longest breath hold (time without inhaling or exhaling) achieved by the user, measures of the regularity of the users breath, measures of the volume produced by a user's breath, measures of the head movement produced by the user during breathing, the time it took users to transition between inhales, exhales, and holds, and detection of which breath stages caused the user to fall out of compliance with an applicable cue.

Summary feedback may further include suggestions for the user to follow in in the future, such as to breathe more deeply, to breathe more shallowly, to take longer breaths, to take shorter breaths, to hold breath longer, to hold breath shorter, to breathe more consistently, to breathe during certain phases of the breath cycle with the nose, and to breathe during certain breath cycles with the mouth. The content and sequencing of cue operations may be determined in advance or at runtime responsive to feedback. For example, in an embodiment wherein a breathwork practice has the goal of gradually reducing a user's breathing rate from their arbitrary baseline breathing rate, cue operations indicating a slower breathing rate than the detected breathing rate of a user in a current or previous session may be used to slow down a user's breathing during the session. In an embodiment that does not include cueing, breathing may be assessed and guidance, feedback, and summary feedback may be presented to the user in a passive breath monitoring mode.

In one embodiment, the method may be implemented on a mixed reality (“MR”) headset equipped with a graphical display and audio output device and worn by the user. Feedback may be given in the form of a stream of 3D particles that proceed from the area of the user's mouth outward (the second object of FIG. 4), representing exhaled breath, then returning to the mouth during inhalation. Feedback is shown when the user's breath matches the cue shown by the first object of FIG. 4 and continues in this fashion as long as the user's respiration is synchronized with the cue. If the detected input indicates that the user is failing to maintain synchronization with the cue, the feedback 3D particles will appear out of alignment with the cue.

In another embodiment, the method may be implemented on a wireless earbud device equipped with pose tracking capabilities and a microphone (a “pose tracking earbud”) or a mobile or desktop application connected to a pose tracking earbud. In such embodiment, feedback is given in the form of audio from the pose tracking earbud or via a visualization of the user's breath displayed inside of a mobile or desktop application. Such a visualization may comprise, for example, a rising and falling ball animation, a growing and contracting circle, etc.—each with the purpose of visualizing the user's breath. If the detected breathing does not match a cue, feedback will show a lack of alignment until synchronization is restored.

An audio component of the system may be used to help guide the user towards correctly completing a breathwork exercise. Breathwork is the regulated control one's breathing to conform to one of a plurality of patterns which are expressed in terms of one or more of: frequency, intensity, rhythm, loudness, sound type, and the movements of specific body parts, such as the nose, mouth, chest, or diaphragm. Using traditional breathwork media, the user is left to breathe with the correct intensity and cadence on their own. Using the system and method described, the audio output and display feedback are provided to the user in real time to guide the user to adapt their breathing. This may come in the form of telling the user to breathe more intensely, focus on a circular cadence, etc.

The system and method disclosed herein can be used with a variety of breathwork exercises.

In some breathwork sessions such as Tummo™ breathing or Wim Hof™ breathing, the user will cyclically hyperventilate and then hold their breath. This produces a sympathetic state for the autonomic nervous system (ANS), referred to as a stress response, which has immune boosting effects and also causes the sensation of focus or clarity for many people. In another form of breathwork, users will emphasize long exhales in order to lower heart rate, stimulating a parasympathetic ANS response. This causes heart rate variability (HRV) to rise which is one way success can be measured in such an exercise. Another breathwork session may include box breathing in which users have equal length inhales, exhales, and holds (as may be present in a square wave, for example).

There are yogic breathwork practices such as ujjayi where users constrict their throat to make a sound that can be focused on during meditation, free-diving breathwork practices, stress reduction breathwork practices, alternate nostril breathing where users breathe with one nostril at a time, etc.

Diaphragmatic breathing means breathing with one's diaphragm and not primarily with the chest. This produces less over breathing (and thus less sympathetic response), fuller breaths, and allows for longer exhales which are helpful for relaxation. Breathing exclusively with the nose has many benefits including aesthetic results due to its impact of facial structure, straightening of teeth due to development of a wider roof of the mouth as a result of nasal breathing, larger airways which can eliminate sleep apnea, and improved athletic performance. The disclosed system's use of audio allows distinguishing between nasal and mouth breathing.

Feedback may further comprise guidance such as corrective feedback, direct instruction, hints, and the like. For example, certain patterns of respiration are dysfunctional or non-adaptive. If a dysfunctional or non-adaptive breathing pattern is identified, this may be communicated to the user. Corrective cues may further be communicated.

A dysfunctional breathing pattern may be specific to a specific Breathwork exercise or may simply mean non-compliance with a given cue, or other non-cued criteria. Specific common examples of dysfunctional breathing in the case of normal breathing where a cue may or may not be omitted (i.e., during rest) include overbreathing (taking too many shallow breaths), mouth breathing (this has negative effects on airway development, teeth alignment, etc.), chest breathing (not breathing with one's diaphragm.

In the case of cyclic hyperventilation, non-adaptive breathing may include stopping inhales or exhales too early or holding one's breath in between inhales and exhales. A common cause of stopping inhales or exhales too early is chest breathing which results in shallow breaths incapable of extending for the desired duration. Thus, a user may be instructed to “breath with their belly” or to “breath deeper”—both types of feedback to help someone breath with their diaphragm.

In the case of a stress reduction breathing exercise with extended exhales, non-adaptive might mean too long of an inhale, too short of an exhale or breathing with the mouth. A user may be instructed to breathe at a certain rate. Dysfunctional may mean not breathing at that rate. In box breathing, dysfunctional may mean not holding after inhales and exhales.

In another breathwork exercise, a user might be instructed to conduct a “two-part inhale” consisting of first breathing with their diaphragm and then their lungs. This double inhale, detectable by our algorithm, could be noticed. A user might fail to comply resulting in feedback reminding them of the cue or providing a more detailed cue about how to achieve a two-part inhale.

An example in a sports performance application comprises the detection of a low breathing rate or irregular cadence and provides real-time feedback to the user to increase the cadence and depth of their breathing in order to produce a sympathetic response and to increase heart rate. This may be done without cueing or may be done in combination with cueing.

Another example usage of the system monitors the characteristics of a user's breathing over a period, assigning a score based on the lowest average breathing rate achieved as well as the consistency of breathing. This may be done without cueing or may be done in combination with cueing. Summary feedback and guidance may be provided immediately following assignment of the score or at a later time.

Another example usage of the system monitors the characteristics of a user's breathing over a period, assigning a score, such as one computed by dividing the total time spent exhaling by the total time spent inhaling. This may be done without cueing or may be done in combination with cueing. Summary feedback and guidance may include such a score.

Some breathwork practices leverage certain characteristics of some embodiments.

In one embodiment which divides the cue operations corresponding to inhale and exhale into three phases: Settle-In, Settled, and Settle-Out (explained in more detail below), a variety of failures can be detected, enabling low-latency, detailed personalized guidance. Assessment during individual phases of a Cyclic Hyperventilation breathwork practice will be used as an example. During the Settle-In phase, if no appreciable motion has been detected, the guidance “do not hold your breath between inhales and exhales” may be given. If motion in the wrong direction is detected during Settle-In, indicating an exhale, the guidance “begin your inhale sooner” may be given. During the Settled phase, if the peak RMS audio intensity is below a threshold, the guidance “breathe more intensely” may be given. If the user enters the Settled phase during inhale or exhale, but they fail to reach an audio intensity threshold or to maintain sufficient motion through to the end of the Settled phase, the guidance “breathe more deeply” or “breathe with your diaphragm” may be given. Summary feedback may include a score, such as one computed by dividing the percentage of breath operations performed by a user matching corresponding cue operations by the total number of cue operations.

In another embodiment which divides the cue operations corresponding to the inhale and exhale into four phases: Settle-In, Settled, Settled-Out, and Hold, a variety of failures can be detected during the individual phases of a Box Breathing Breathwork Practice, enabling low-latency, detailed personalized guidance. For example, if a user stops their breath hold too early as detected by motion or RMS audio intensity above a threshold at the end of the Hold phase guidance such as “focus on maintaining equal length holds, inhales, and exhales” may be given. In another example, if a user begins their breath hold late as detected by motion above a threshold at the beginning of the Hold phase, guidance such as “extend your exhale,” “extend your inhale,” and the like may be given.

In another embodiment which divides the cue operations corresponding to the inhale and exhale into five phases: Settle-In, Settled-Diaphragm, Transition, Settled-Chest, Settle-Out (explained in more detail below), a variety of failures can be detected during the individual phases of a Stress Reduction breathwork practice, enabling low-latency, detailed personalized guidance. For example, if a user fails to enter the Transition phase, the detection of which is described below, guidance such as “Place one hand on your belly and one hand on your chest. Feel your belly rise before your chest.” may be given.

In some embodiments, a cue operation may instruct the user to make a certain kind of sound when breathing, compliance with that instruction can be assessed by observing the peak RMS audio intensity during the Settled phase and giving guidance such as “focus on the sounds of your exhale.” Some embodiments compute a fast Fourier transform (FFT) of the audio waveform to determine whether the user produces the sound required by the exercise. Different feedback and guidance may be provided based on the detected breathing. In one example, where a user is cued not to hold breath between inhales and exhales during a cyclical hyperventilation, Tummo™, or Wim Hof™ breathwork session, guidance may include, for example, “keep your breathing circular,” “imagine your breath as a wave,” “do not hold your breath between inhales and exhales.” In a further example, where the user is determined to have had a prolonged exhale overlapping the time a user is supposed to begin inhale, guidance may include “follow the cue,” “begin your inhale sooner”. In a situation where the user fails to breathe loud enough during a cyclical hyperventilation, Tummo™, or Wim Hof™ breathwork session, guidance may include “breath more intensely”. In a situation where the user is detected as failing to follow an instruction to produce a certain tone during exhale during a stress reduction extended exhale session, guidance may include “remember to purse your lips and make a hissing sound to slow your exhale.” In yet another example, where a user is determined to have erratic breathing, guidance may comprise “return your focus to your breath,” or “follow the cue.”

In some embodiments, feedback can be delivered to instruct the user to adjust their breathing for the purpose of causing an increase or decrease in heart rate. Short, sharp inhales increase heart rate while long slow exhales lower heart rate. The duration and speed of inhales may be detected and guidance provided to adjust the speed and duration of inhales and exhales to a given range until heart rate is in an optimal performance zone. This may be done without cueing or may be done in combination with cueing. Measurement of heart rate may be done in any suitable manner, including a wearable heart rate monitor (HRM).

In another embodiment, the system and method may use the ratio of detected exhale depth to inhale depth, as determined by the tracking module (FIGS. 1a and 1b), to produce a measurement of stress for the user, a low exhale depth to inhale depth ratio may indicate a relatively high stress state. This may be done without cueing or may be done in combination with cueing. Guidance may comprise “take it easy today,” or “try to make time to recharge.”

### Determining Breath State

The system and method described herein measure user movement and audio to determine breathing pattern. This involves capturing and isolating movement, capturing audio, establishing a baseline, tracking and smoothing pose, and determining breath state. It is to be appreciated that some of these steps may be omitted and/or additional steps may be included.

In general, the system and method measure and assess breath independent of constraints such as head movement at a certain speed or in a certain direction. Each of the positional and rotational elements of pose may be observed and measured. The system and method observe, determine, and iteratively update dynamic values including, optionally, baselines, identify when the user crosses the baselines, and/or when the user changes direction. Breath detection is based on these baseline-crossings and/or direction changes, optionally correlated with audio input, and the system and method are therefore able to track the user's breathing regardless of how frequently or how deeply they are breathing. This is an advantage over systems that detect only whether or not the user is conforming to given cue operations, and systems that only detect breathing rate, because it can provide breath visualization independent of conformance to cue operations. This comprises one type of low-latency guidance about how to move into conformance with cue operations, for example.

### Isolating Movement

To monitor and track respiration, various movements may be monitored. These may include, for example, movement of the head, movement of other body parts, and facial deformation. Tracking of some movements may involve tracking individual contributions from three axes of positional displacement and three axes of rotation.

Commonly available systems pre-process and combine sensor data into world-space coordinates. In various embodiments, this process may be omitted or undone. Consider a local coordinate system (Head Space) that is aligned according to FIG. 5a such that, from the user's perspective, the z-axis is longitudinal, the x-axis is lateral, and the y-axis is vertical; this is the desired frame of reference, and sensor data should be made available in this format. In systems where these values are not conflated, for example, direct readings from a head-aligned accelerometer, separation would be unnecessary but calculations including without limitation integration of acceleration data may be required. Such systems may include any number of commercial head-mounted displays, glasses, masks, visors, wireless earbuds, or other accessories.

In an embodiment comprising a MR headset or Pose Tracking Earbud worn by the user, a plurality of sensors are provided in the headset or Pose Tracking Earbud. The sensors may include accelerometers and/or gyroscopes and may provide pose measurements of a physical body over time. Referring to FIGS. 5a and 5b, at the time (τ) of each successive measurement, the displacement of the head in world space is determined by subtracting the previously recorded position (P_{τ-1}) from the current position (P_{τ}); this produces a displacement (ΔP). The orientation of Head Space is represented by a quaternion (Q_{τ}). In order to rotate ΔP to Head Space, ΔP is multiplied by the inverse of Q_{τ}, producing P′_{τ}. The individual components of motion are then extracted with vector projection: P′_{τ} is projected onto vectors [1,0,0], [0,1,0], and [0,0,1] to obtain lateral, vertical, and longitudinal movement, respectively. Additionally, rotational changes of a user's head can be used for breath tracking. Referring to FIG. 5c, one can capture the difference in orientation of a user's head between time τ−1 and time τ.

In some embodiments, the system may include a camera. In some embodiments, an external camera (meaning a camera separated from the user) may be used. Such cameras may comprise a smartphone or tablet. The camera may be positioned to the side of the user to capture imagery from which vertical and longitudinal movement of the user's head can be extracted using computer vision algorithms. The camera may further be used to track additional body movements, such as chest expansion and contraction and shoulder and neck movements. Such a camera may be used with a head-mounted device such as a MR headset or Pose Tracking Earbud. Alternatively, such a camera may be used without a head-mounted device, making the process less intrusive and more accessible for users.

Facial deformation is another bodily attribute that correlates with breathing. 3D facial recognition technology such as stereo cameras and structured-light-based systems can be used to capture changes in facial geometry that correlate with the different stages of the breath cycle. Similarly, 3D imaging technologies may be used to capture changes in the geometry of the chest and abdomen that correlate with breathing. Force sensors, strain gauges, or other similar sensors could also be used to detect the deformation of the nose resultant from nasal breathing.

External depth-sensing technologies, including without limitation passive infrared (“PIR”), light detection and ranging (“LIDAR”), and ultrasonic sensors, can be used independently or in conjunction with the aforementioned technologies to measure respiratory-induced body movements.

### Filtering

In some embodiments, such as where a plurality of sensors are used, and where noise is present, from extraneous factors which are high frequency relative to respiration, a low-pass filter is helpful in conditioning the data for real-time analysis. Some factors that may contribute to noise include spurious body movement, overly sensitive measuring equipment, sensor shifting relative to the subject, or high-frequency bodily events.

Exponential smoothing (also called “alpha blending”) is suitable for smoothing or filtering the data. Other filter types may be equally suitable and may alternatively be used, including, for example, Kalman filter, Butterworth filter, Bessel filter, Optimum “L” (Legendre) filter, and Linkwitz-Riley filter. To smooth an input sample (“i_{τ}”), a value (“α”) between zero and one, inclusive, is chosen. The filtered value (“f_{τ}”) is calculated as f_{τ}=(α)*i_{τ}+(1−α)*f_{τ-1}. α can be tuned to optimize the degree to which higher frequency components are damped.

FIG. 4 illustrates a sinusoidal movement with noise and the same data after exponential smoothing. Specifically, the top of FIG. 4 illustrates the sinusoidal movement as detected and the bottom of FIG. 4 illustrates the sinusoidal movement detected after exponential smoothing (α=0.01).

### Audio

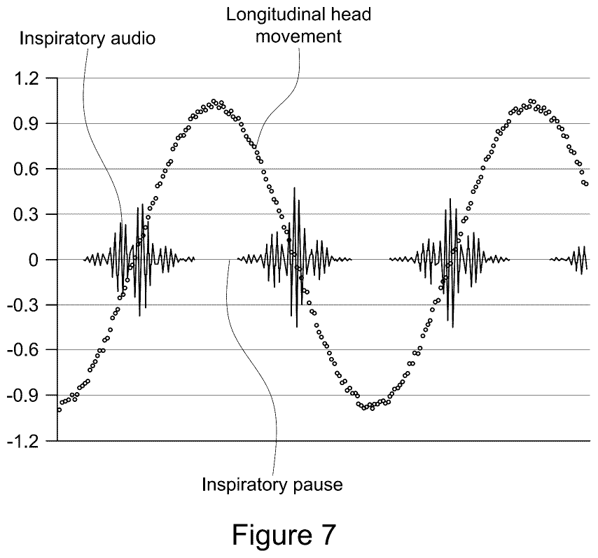

FIG. 7 illustrates acoustic input from respiration in relation to head movement. Acoustic samples can be captured and used to improve the system's accuracy by, for example, comparing the strength of an audio signal caused by respiration to a threshold value. The audio input can be used in isolation or in combination with other inputs (e.g. movement) when tracking a breath cycle, as shown. Some methods, such as cross-correlation, Fourier analysis, or Machine Learning, may be used on their own or be used in combination with one another or traditional signal processing techniques, to identify each phase of a breath cycle, independent of user movement.

In one embodiment of the present invention, acoustic samples originating from the subject's nose and mouth are captured. A Root Mean Square (RMS) value is calculated over the range of samples. The RMS is compared to a threshold value, above or below which an estimate may be made of the likelihood that the user is actively inhaling, exhaling, or holding their breath. RMS audio intensity can be observed over time and the shape of the intensity curve used to determine a specific breath state, such as inhale or exhale

Several other optional steps may be taken to increase the accuracy of this estimate in the presence of ambient noise. Example steps include noise removal algorithms utilizing software and/or hardware, dynamic adjustment of threshold parameters, and/or static threshold parameters calibrated to detect respiratory events above the noise floor.

### Baseline

The system and method provided herein can establish a baseline and continually adjust a baseline during breathing routines.

Respiration can be roughly modeled as a periodic function including but not limited to a sine wave, a trapezoidal wave, or a quasi-square wave that crosses back and forth over a neutral midpoint. Unlike a periodic function, however, a user's bodily position in space is arbitrary, and this neutral midpoint is unknown. Such a periodic function is said to have a DC bias. A waveform that is free of this bias is said to be “DC-balanced,” which is a desirable property when calculating amplitude, for example. To arrive at a DC-balanced wave, the system and method remove the bias and establish an estimate of this neutral point, referred to as a baseline. The baseline may be continually adjusted using software algorithms and/or hardware components. This may be useful in scenarios where the user changes their position by a significant amount (more than would be observed by mere respiration).

Because a baseline is derived from the value being observed, a separate baseline may be derived for each independent axis or dimension of measurement, such as rotation, position, and/or RMS audio.

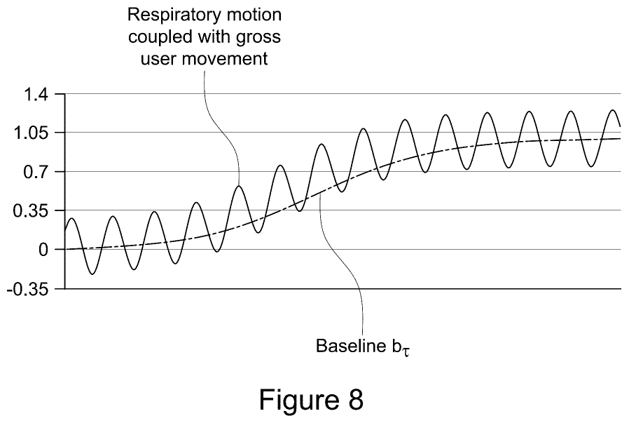

In some embodiments, a plurality of baselines are calculated. FIG. 8 illustrates a baseline tracking the neutral midpoint of periodic motion even as a large offset is introduced (α=0.06). The system and method take isolated, periodic spatial components and apply exponential smoothing (as with fₜ above) to the degree that an overdamped wave (bₜ) is produced for each axis. The α parameter is dynamic in baseline calculations (unlike fₜ above). A deadzone is established above and below a baseline such that small movements observed during respiration remain in this region. α is constant as long as movement falls within the deadzone such that the baseline is unaffected by normal breathing. Movement outside of the deadzone makes the value of a inversely proportional to the user's change in position; a large change will reduce damping and allow a significant correction to this baseline. A baseline will stabilize to a close approximation of its true neutral midpoint once the user is stationary, even while breathing. Subtracting the baseline value from the input (f_{τ}-b_{τ}) yields a DC-balanced waveform suitable for tracking the components of movement of the head resulting from respiration and not resulting from, for example, body movement.

### Pose Tracking

Respiration generally causes detectable movement of the head. For example, a stationary user in an upright sedent position engaged in techniques such as “cyclic hyperventilation” or “box breathing” will typically experience upward and backward movement of the head during inhalation and the opposite during exhalation. Using isolated spatial components of the pose relative to respective baseline values, the inspiratory and expiratory phases of the breath cycle can be inferred. Other patterns of breathing have varied changes in pose that can be tracked using the same method.

Respiration also produces rotations around the x, y, and z axes. These rotations are commonly referred to as pitch, yaw, and roll respectively. With reference to the cyclic hyperventilation technique, respiration causes a detectable amount of pitch, though the direction of pitch at each phase differs between subjects. For example, during inhalation, one person may pitch their head upward, while another tends downward. This inconsistency can be overcome in several ways, including but not limited to a calibration step, user configuration, or automatic detection of the specific (direct or inverse) behavior by correlating pitch changes with linear head movement.

Referring back to FIG. 7, audio input can reinforce pose-based breath cycle detection, as characteristic sound levels are generated at each phase of the cycle, indicating inhalation, exhalation, or inspiratory/expiratory pause/hold. Audio input frequency and time domain characteristics may also be used to identify stages of the breath cycle and may further reinforce pose-based cycle detection.

Some users may have atypical pose changes. The respiration of these users may be better tracked using Machine Learning. By collecting user pose and audio information and correlating it to the breath cues, one can accurately classify the breath patterns of a user and adapt the algorithm to those patterns.

In one embodiment, breath state, described below, may be determined by comparing elements of pose (the vector components of position and orientation in Head Space) to their respective baselines. For example, pose may be considered to have reached a peak when it first crosses the baseline and then changes direction. Based on the direction of movement before and after hitting a peak, an exhale to baseline or inhale to baseline state is assigned. Similarly, the direction of movement during a baseline crossing determines the assignment of exhale to peak or inhale to peak states. In another embodiment, breath state may be determined by direction changes without reference to a baseline

### Breath State

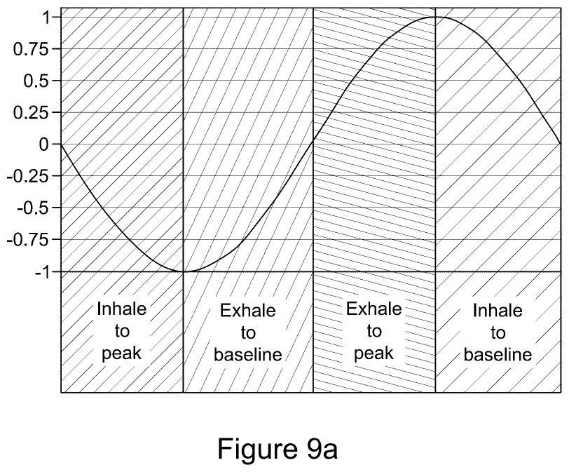

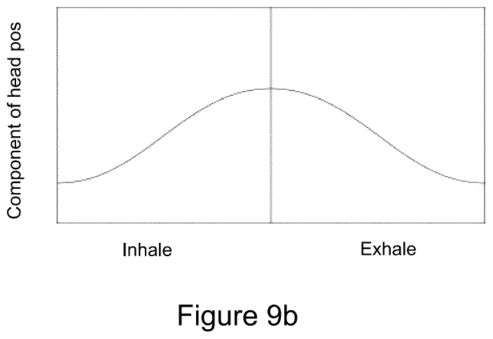

FIG. 9a illustrates one embodiment in which four phases of motion are tracked during respiration in a Cyclic Hyperventilation: inhale to peak (“IP”), exhale to baseline (“EB”), exhale to peak (“EP”), and inhale to baseline (“IB”).

FIG. 9b illustrates another embodiment which omits the use of baselines and uses only direction changes to track two phases of motion: inhale to peak (“IP”) and exhale to peak (“EP”).

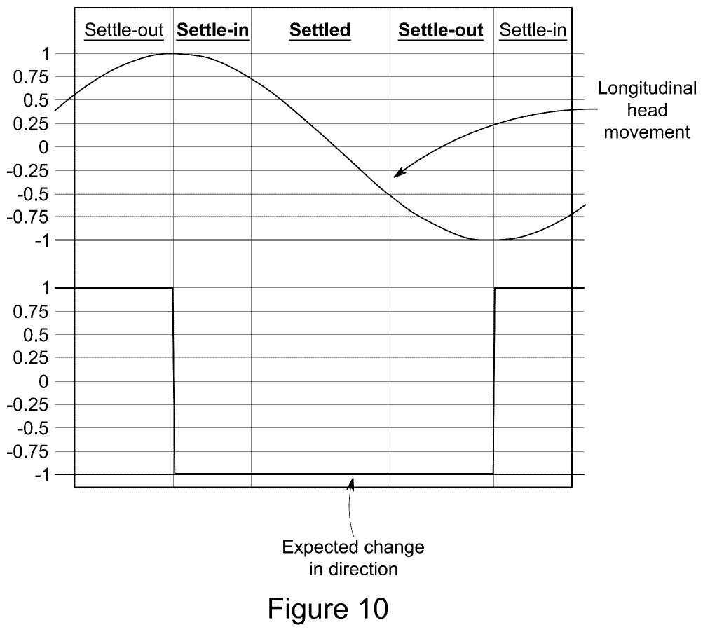

The time between each cue operation (for example, an instruction to inhale, to exhale, or to hold) may be divided into three phases or time windows: Settle-in, Settled, and Settle-out, shown in FIG. 10. The duration of each phase may be predetermined and prescribed to the user or dynamically determined during use. In some embodiments, the durations of Settle-in and Settle-out may be empirically determined optimal constants suitable for most users to transition into or out of inhalation or exhalation. In other embodiments, the durations of Settle-in and Settle-out may be computed responsive to the user's observed breathing patterns, in real time or based on summary feedback. In various embodiments, optimization may be performed to, for example, improve user compliance with cues.

An example of process flow through these phases is described. This is for exemplary purposes only. During Settle-in, when a user is to begin an inhalation or exhalation, the state indicated by pose tracking must become consistent with that of the given cue operation type. For example, it is expected that during an inhale cue, pose tracking would report either the IB or IP states. For an exhale cue, EB or EP would be expected. In addition to pose tracking, the RMS audio level may be required to meet or exceed the required threshold to be considered valid breath input. If these conditions are met before the expiration of the Settle-in window, the system and method continue to the Settled window, otherwise, the user is considered out of sync and the system waits for the next cue operation signal. During the Settled window, in which the user is following through with inhalation or exhalation, the state or motion indicated by pose tracking must continue to match that of the cue operation type. If the Settled window expires and the pose tracking state is maintained, the Settle-out window is entered, in which the user is approaching an inspiratory or expiratory pause or change. As before, if the user fails to maintain consistent movement and/or RMS audio level before the expiration of the Settled window, they are out of sync and the next cue operation is awaited. No conditions are required to pass through the Settle-out window, and they are considered fully synchronized with the system.

A variation of this embodiment uses the time recorded at each breath state transition to calculate the length of the current quarter cycle. By observing the relative durations of the four states, instantaneous breaths per minute (“BPM”) can be estimated at the end of each state. When a transition is made out of either peak state, a signal (“Hit Peak”) is generated to indicate that the flow of breath has reversed from inhale to exhale or vice versa. This can also be done using the two state approach of IP and EP.

In another embodiment in which the user is instructed to inhale in two phases consisting first of a diaphragmatic inhale (“DI”) followed by an inhale using the chest (“CI”), known as a “two-part inhale,” the transition between the DI and CI is detected by observing a local minimum in one or both of audio input or pose change. In this embodiment, the following breath states are maintained: Inhale to Peak (“IP”), Exhale to Baseline (“EB”), Exhale to Peak (“EP”), and Inhale to Transition (“IT”). The inhale process flow in this embodiment would be Settle-In, Settled-Diaphragm, Transition, Settled-Chest, Settle-Out. The exhale process flow is the same as that of the previously-described embodiment.

In another embodiment in a pose tracking schema that includes a breath hold state, if the cue operation is a breath hold, pose tracking may be used to enforce minimal movement during the cue operation, as no particular type of movement is necessarily expected from the user during that time. The RMS audio level can optionally be monitored to determine if a premature inhale/exhale has occurred.

### Optimization

Optimization is the process of improving the detection of compliance and non-compliance with an applicable cue operation during breathwork or other traits of a user's breathing with or without the presence of a cue, such as frequency, intensity, etc. Optimization includes using algorithms that search for optimal values used as input to breath detection methodologies. In the case of breathwork, values obtained via optimization may differ depending on the exercise which is being optimized. Optimization can be performed using collected data from users as well as in real time. Data used for optimization includes head pose, audio RMS intensity, etc.

In one embodiment, optimization determines the values for a baseline and smoothing alpha. A space of values for these variables is searched such that those chosen values result in the most accurate breath detection for the input data.

In another embodiment, optimization determines the values for a smoothing alpha. A space of values for this variable is searched such that those chosen values result in the most accurate breath detection for the input data.

In another embodiment, optimization determines the smallest possible Settle-In and Settle-Out values. This looks to minimize the length of these windows so the compliance of a user is ensured for a larger percentage of the breath duration.

In another embodiment, optimization determines which form of data is best able to detect breath compliance and non-compliance. The optimizer may choose one or more of: 3 axes of positional displacement, 3 axes of rotation, and RMS audio intensity. This includes the axes of motion, axes of rotation, microphone RMS intensity, etc.

In another embodiment, optimization determines the efficacy of recorded data which may or may not be included in further optimization. This embodiment examines each recorded session and determines whether or not it conforms to the breath pattern expected in the exercise being optimized.

In another embodiment, optimization is done in real time, such that the breath detection algorithm can make adjustments based on the current user's data. For example, the optimization techniques described above may be used incrementally after each breath cycle to update smoothing alphas, baseline alphas, and Settle-In/Settle-Out values.

### Use Cases

The system and method provided herein may be used in any suitable application. Such applications include, for example, health and performance as well as entertainment.

Health and performance uses include use with biometric feedback and wearables, stress regulation, tactical simulation, and health applications, for example. In use with biometric feedback and wearables, the system and method track breath efficiency for mindfulness and performance training. This can be done using consumer wearables. In stress regulation, the system and method may be used go guide breathing exercises with real-time feedback. The system and method may be used for stress management in military, law enforcement, pilot training, and the like. In health applications, the system and method may be used for respiratory tracking and integration with wearables.

Entertainment uses include realism in gaming, breath-controlled interactions, and cinematic audio experiences, for example. For realism in gaming, the system and method may be used to adjust difficulty of cognitive training and gaming based on breath state (e.g. shooting a firearm, seeing your breath). For breath-controlled interactions, the system and method may be used to provide real-time visualization of breathing. In cinematic audio experiences, the system and method may adjust soundtrack intensity or surround sound effects in response to breath rate, creating a dynamic and personalized experience.

### Miscellaneous

For purposes of this disclosure, any system described herein may include any instrumentality or aggregate of instrumentalities operable to compute, calculate, determine, classify, process, transmit, receive, retrieve, originate, switch, store, display, communicate, manifest, detect, record, reproduce, handle, or utilize any form of information, intelligence, or data for business, scientific, control, or other purposes. For example, a system or any portion thereof may be a wearable device, minicomputer, mainframe computer, personal computer (e.g., desktop or laptop), tablet computer, embedded computer, mobile device (e.g., personal digital assistant (PDA) or smartphone) or other hand-held computing device, server (e.g., blade server or rack server), a network storage device, or any other suitable device or combination of devices and may vary in size, shape, performance, functionality, and price. A system may include volatile memory (e.g., random access memory (RAM)), one or more processing resources such as a central processing unit (CPU) or hardware or software control logic, ROM, and/or other types of nonvolatile memory (e.g., EPROM, EEPROM, etc.). A basic input/output system (BIOS) can be stored in the non-volatile memory (e.g., ROM), and may include basic routines facilitating communication of data and signals between components within the system. The volatile memory may additionally include a high-speed RAM, such as static RAM for caching data.

Additional components of a system may include one or more disk drives or one or more mass storage devices, one or more network ports for communicating with external devices as well as various input and output (I/O) devices, such as digital and analog general purpose I/O, a keyboard, a mouse, touchscreen and/or a video display. Mass storage devices may include, but are not limited to, a hard disk drive, floppy disk drive, CD-ROM drive, smart drive, flash drive, or other types of non-volatile data storage, a plurality of storage devices, a storage subsystem, or any combination of storage devices. A storage interface may be provided for interfacing with mass storage devices, for example, a storage subsystem. The storage interface may include any suitable interface technology, such as EIDE, ATA, SATA, and IEEE 1394. A system may include what is referred to as a user interface for interacting with the system, which may generally include a head-mounted display, earbuds, display, mouse or other cursor control device, keyboard, button, touchpad, touch screen, stylus, remote control (such as an infrared remote control), microphone, camera, video recorder, gesture systems (e.g., eye movement, head movement, etc.), speaker, LED, light, joystick, game pad, switch, buzzer, bell, and/or other user input/output device for communicating with one or more users or for entering information into the system. These and other devices for interacting with the system may be connected to the system through I/O device interface(s) via a system bus, but can be connected by other interfaces such as a parallel port, IEEE 1394 serial port, a game port, a USB port, an IR interface, Bluetooth, Wi-Fi (wireless fidelity), etc. Output devices may include any type of device for presenting information to a user, including but not limited to, a computer monitor, flat-screen display, or other visual display, a printer, and/or speakers or any other device for providing information in audio form, such as a telephone, a plurality of output devices, or any combination of output devices.

A system may also include one or more buses operable to transmit communications between the various hardware components. A system bus may be any of several types of bus structure that can further interconnect, for example, to a memory bus (with or without a memory controller) and/or a peripheral bus (e.g., PCI, PCIe, AGP, LPC, I2C, SPI, USB, etc.) using any of a variety of commercially available bus architectures.

One or more programs or applications, such as a web browser and/or other executable applications, may be stored in one or more of the system data storage devices. Generally, programs may include routines, methods, data structures, other software components, etc., that perform particular tasks or implement particular abstract data types. Programs or applications may be loaded in part or in whole into a main memory or processor during execution by the processor. One or more processors may execute applications or programs to run systems or methods of the present disclosure, or portions thereof, stored as executable programs or program code in the memory, or received from the Internet or other network. Any commercial or freeware web browser or other application capable of retrieving content from a network and displaying pages or screens may be used. In some embodiments, a customized application may be used to access, display, and update information. A user may interact with the system, programs, and data stored thereon or accessible thereto using any one or more of the input and output devices described above.

A system of the present disclosure can operate in a networked environment using logical connections via a wired and/or wireless communications subsystem to one or more networks and/or other computers. Other computers can include, but are not limited to, workstations, servers, routers, personal computers, microprocessor-based entertainment appliances, peer devices, or other common network nodes, and may generally include many or all of the elements described above. Logical connections may include wired and/or wireless connectivity to a local area network (LAN), a wide area network (WAN), hotspot, a global communications network, such as the Internet, and so on. The system may be operable to communicate with wired and/or wireless devices or other processing entities using, for example, radio technologies, such as the IEEE 802.xx family of standards, and includes at least Wi-Fi (wireless fidelity), WiMax, and Bluetooth wireless technologies. Communications can be made via a predefined structure as with a conventional network or via an ad hoc communication between at least two devices.

Hardware and software components of the present disclosure, as discussed herein, may be integral portions of a single computer, server, controller, or message sign, or may be connected parts of a computer network. The hardware and software components may be located within a single location or, in other embodiments, portions of the hardware and software components may be divided among a plurality of locations and connected directly or through a global computer information network, such as the Internet. Accordingly, aspects of the various embodiments of the present disclosure can be practiced in distributed computing environments where certain tasks are performed by remote processing devices that are linked through a communications network. In such a distributed computing environment, program modules may be located in local and/or remote storage and/or memory systems.

As will be appreciated by one of skill in the art, the various embodiments of the present disclosure may be embodied as a method (including, for example, a computer-implemented process, a business process, and/or any other process), apparatus (including, for example, a system, machine, device, computer program product, and/or the like), or a combination of the foregoing. Accordingly, embodiments of the present disclosure may take the form of an entirely hardware embodiment, an entirely software embodiment (including firmware, middleware, microcode, hardware description languages, software subscriptions, app subscriptions, etc.), or an embodiment combining software and hardware aspects. Furthermore, embodiments of the present disclosure may take the form of a computer program product on a computer-readable medium or computer-readable storage medium, having computer-executable program code embodied in the medium, that define processes or methods described herein. A processor or processors may perform the necessary tasks defined by the computer-executable program code. Computer-executable program code for carrying out operations of embodiments of the present disclosure may be written in an object oriented, scripted or unscripted programming language such as Java, Perl, PHP, Visual Basic, Smalltalk, C++, or the like. However, the computer program code for carrying out operations of embodiments of the present disclosure may also be written in conventional procedural programming languages, such as the C programming language or similar programming languages. A code segment may represent a procedure, a function, a subprogram, a program, a routine, a subroutine, a module, an object, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted via any suitable means including memory sharing, message passing, token passing, network transmission, etc.

In the context of this document, a computer readable medium may be any medium that can contain, store, communicate, or transport the program for use by or in connection with the systems disclosed herein. The computer-executable program code may be transmitted using any appropriate medium, including but not limited to the Internet, optical fiber cable, radio frequency (RF) signals or other wireless signals, or other mediums. The computer readable medium may be, for example but is not limited to, an electronic, magnetic, optical, electromagnetic, infrared, or semiconductor system, apparatus, or device. More specific examples of suitable computer readable medium include, but are not limited to, an electrical connection having one or more wires or a tangible storage medium such as a portable computer diskette, a hard disk, a random access memory (RAM), a read-only memory (ROM), an erasable programmable read-only memory (EPROM or Flash memory), a compact disc read-only memory (CD-ROM), or other optical or magnetic storage device. Computer-readable media includes, but is not to be confused with, computer-readable storage medium, which is intended to cover all physical, non-transitory, or similar embodiments of computer-readable media.

Various embodiments of the present disclosure may be described herein with reference to flowchart illustrations and/or block diagrams of methods, apparatus (systems), and computer program products. It is understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks. Alternatively, computer program implemented steps or acts may be combined with operator or human implemented steps or acts in order to carry out an embodiment of the invention.

Additionally, although a flowchart or block diagram may illustrate a method as comprising sequential steps or a process as having a particular order of operations, many of the steps or operations in the flowchart(s) or block diagram(s) illustrated herein can be performed in parallel or concurrently, and the flowchart(s) or block diagram(s) should be read in the context of the various embodiments of the present disclosure. In addition, the order of the method steps or process operations illustrated in a flowchart or block diagram may be rearranged for some embodiments. Similarly, a method or process illustrated in a flow chart or block diagram could have additional steps or operations not included therein or fewer steps or operations than those shown. Moreover, a method step may correspond to a method, a function, a procedure, a subroutine, a subprogram, etc.

As used herein, the terms “substantially” or “generally” refer to the complete or nearly complete extent or degree of an action, characteristic, property, state, structure, item, or result. For example, an object that is “substantially” or “generally” enclosed may mean that the object is either completely enclosed or nearly completely enclosed. The exact allowable degree of deviation from absolute completeness may in some cases depend on the specific context. However, generally speaking, the nearness of completion will be so as to have generally the same overall result as if absolute and total completion were obtained. The use of “substantially” or “generally” is equally applicable when used in a negative connotation to refer to the complete or near complete lack of an action, characteristic, property, state, structure, item, or result. For example, an element, combination, embodiment, or composition that is “substantially free of” or “generally free of” an element may still actually contain such element as long as there is generally no significant effect thereof.

To aid the Patent Office and any readers of any patent issued on this application in interpreting the claims appended hereto, applicants wish to note that they do not intend any of the appended claims or claim elements to invoke 35 U.S.C. § 112(f) unless the words “means for” or “step for” are explicitly used in the particular claim.

Additionally, as used herein, the phrase “at least one of [X] and [Y],” where X and Y are different components that may be included in an embodiment of the present disclosure, means that the embodiment could include component X without component Y, the embodiment could include the component Y without component X, or the embodiment could include both components X and Y. Similarly, when used with respect to three or more components, such as “at least one of [X], [Y], and [Z],” the phrase means that the embodiment could include any one of the three or more components, any combination or sub-combination of any of the components, or all of the components.

In the foregoing description various embodiments of the present disclosure have been presented for the purpose of illustration and description. They are not intended to be exhaustive or to limit the invention to the precise form disclosed. Obvious modifications or variations are possible in light of the above teachings. The various embodiments were chosen and described to provide the best illustration of the principals of the disclosure and their practical application, and to enable one of ordinary skill in the art to utilize the various embodiments with various modifications as are suited to the particular use contemplated. All such modifications and variations are within the scope of the present disclosure as determined by the appended claims when interpreted in accordance with the breadth they are fairly, legally, and equitably entitled.

What is claimed is:

1. A method for assessing breathing comprising:
sensing input about a user's breathing over a time period;
gathering data about the user's breathing;
assessing the user's breathing; and
providing feedback to the user regarding the user's breathing.

2. The method of claim 1, wherein sensing input comprises gathering data about the user's breathing over the time period.

3. The method of claim 2, wherein the data gathered is about movement of the user's head and/or thoracic region during the time period.

4. The method of claim 2, wherein the data gathered includes pose.

5. The method of claim 4, wherein the pose comprises position and orientation of the user's head.

6. The method of claim 1, wherein the data gathered includes sounds produced by the user's body.

7. The method of claim 6, wherein the sounds include sounds produced by the user's mouth and/or sounds produced by the user's nose.

8. The method of claim 1, wherein the feedback includes a score.

9. The method of claim 1, wherein the feedback is one of “breathe more quickly,” “breathe more slowly,” “breathe more deeply,” “extend your inhale,” or “extend your exhale.”

10. The method of claim 1, further comprising providing a cue to the user prior to gathering data about the user's breathing, wherein the cue is one of a cue type.

11. The method of claim 10, wherein the cue type is one of inhale, exhale, or hold.

12. The method of claim 10, wherein assessing the user's breathing comprises assessing compliance with the cue type.

13. The method of claim 12, wherein assessing compliance with cue type includes determining a breath state of the user and comparing the breath state to the cue type.

14. The method of claim 10, wherein the feedback includes a score and wherein the score is calculated by dividing a percentage of breath operations performed by a user matching corresponding cues by a total number of cues.

15. The method of claim 1, further comprising establishing a baseline for elements of the user's pose.

16. The method of claim 15, further comprising iteratively adjusting the baseline during the user's breathing.

17. A system for assessing a user's breathing, the system comprising:
one or more pose tracking systems for tracking breathing of a user;
a tracking module for collecting data about the user's breathing from the one or more pose tracking systems;
an assessment module for assessing the user's breathing; and
a feedback module for providing feedback to the user regarding the user's breathing.

18. The system of claim 17, wherein the one or more pose tracking systems detect at least one of position, positional acceleration, rotation, and rotational acceleration of user's head and/or thoracic region.

19. The system of claim 18, wherein at least one of the one or more the pose tracking systems is disposed in a wearable or in a virtual or augmented reality headset.

20. The system of clam 17, further comprising a microphone, wherein the microphone tracks audio associated with breathing of the user.
